SEQUENCE LISTING (21 sequences)

SEQ ID NO: 1 - DNA, 154 bp, Influenza A virus
aaagggaaagtggttaaatcagtcgaattggatgctcctaattatcactatgaggagtgctcctgttatccttttgatgccggcgaaatcacatgtgtgtgcagggataattggcatggctcaaataggccatgggtatctttcaatcaaaatt

SEQ ID NO: 2 - DNA, 22 bp, Artificial
  Primer sequence
agtcgaattggatgctcctaat

SEQ ID NO: 3 - DNA, 17 bp, Artificial
  Primer sequence
gcctatttgagccatgc

SEQ ID NO: 4 - DNA, 27 bp, Artificial
  Probe sequence
aggagcactcctcatagtgataattag

SEQ ID NO: 5 - DNA, 20 bp, Artificial
  Prober sequence
gtgcagggataattggcatg

SEQ ID NO: 6 - DNA, 20 bp, Artificial
  Primer sequence
agtcgaattggatgctccta

SEQ ID NO: 7 - DNA, 18 bp, Artificial
  Primer sequence
cccatggcctatttgagc

SEQ ID NO: 8 - DNA, 29 bp, Artificial
  Probe sequence
ggataacaggagcactcctcatagtgata

SEQ ID NO: 9 - DNA, 22 bp, Artificial
  Probe sequence
gtgtgtgcagggataattggca

SEQ ID NO: 10 - DNA, 25 bp, Artificial
  Primer sequence
gaattggatgctcctaattatcact

SEQ ID NO: 11 - DNA, 21 bp, Artificial
  Primer sequence
ctatttgagccatgccaatta

SEQ ID NO: 12 - DNA, 21 bp, Artificial
  Primer sequence
tagagttgaatgcacccaatt

SEQ ID NO: 13 - DNA, 19 bp, Artificial
  Primer sequence
aggtcgatttgaaccatgc

SEQ ID NO: 14 - DNA, 29 bp, Artificial
  Probe sequence
ggaacattcctcataatgaaaattgggtg

SEQ ID NO: 15 - DNA, 17 bp, Artificial
  Probe sequence
cagggacaactggcatg

SEQ ID NO: 16 - DNA, 21 bp, Artificial
  Primer sequence
caatagagttgaatgcaccca

SEQ ID NO: 17 - DNA, 21 bp, Artificial
  Primer sequence
ccaaggtcgatttgaaccatg

SEQ ID NO: 18 - DNA, 31 bp, Artificial
  Probe sequence
ctgggtaacaggaacattcctcataatgaaa

SEQ ID NO: 19 - DNA, 25 bp, Artificial
  Probe sequence
tgatgtgtgtatgcagggacaactg

SEQ ID NO: 20 - DNA, 22 bp, Artificial
  Primer sequence
ttgaatgcacccaattttcatt

SEQ ID NO: 21 - DNA, 20 bp, Artificial
  Primer sequence
gatttgaaccatgccagttg

(19) United States
(12) Patent Application Publication
(10) Pub. No.: US 2013/0210001 A1
(43) Pub. Date: Aug. 15, 2013

(54) SEQUENCE DETECTION ASSAY

(71) Applicant: Martin Lee, Wiltshire (GB)

(72) Inventor: Martin Lee, Wiltshire (GB)

(73) Assignee: ENIGMA DIAGNOSTICS LIMITED, Wiltshire (GB)

(21) Appl. No.: 13/634,925

(22) PCT Filed: Mar. 15, 2011
(86) PCT No.: PCT/GB2011/050508

§ 371 Date: Apr. 22, 2013

(30) Foreign Application Priority Data
Mar. 16, 2010 (GB) 1004339.6

(51) Int. Cl. C12Q 1/68

(52) U.S. Cl. CPC C12Q 1/6818
USPC 435/6.11

(57) ABSTRACT

There is provided a method of detecting the presence in a sample of a first target sequence and a second target sequence within a test region of a nucleic acid sequence comprising conducting a nucleic acid amplification reaction, to form a forward amplicon strand and a reverse amplicon strand of the test region, contacting the forward amplicon strand with a first probe labelled with a first FRET label and capable of hybridising to the first target sequence of complement thereof in the forward amplicon strand, and contacting the reverse amplicon strand with a second probe labelled with a second FRET label and capable of hybridising to the second target sequence or complement thereof in the reverse amplicon strand; wherein the nucleic acid amplification reaction is conducted using a forward amplification primer labelled with a third FRET label and a reverse amplification primer labelled with a fourth FRET label, the forward primer being incorporated into the forward amplicon strand and the second primer being incorporated into the reverse amplicon strand during the amplification reaction; and further wherein the first and third FRET labels form a FRET pair and the second and fourth FRET labels form a different FRET pair, each FRET pair comprising a donor label; the method further comprising the steps of exposing the sample to an excitation source having a wavelength which excites the donor label in the first FRET pair and the donor label in the second FRET pair, detecting fluorescence from the sample and relating this to the presence or absence of the first and second target sequences.

### FIELD OF INVENTION

The present invention provides a method for detecting more than one target sequence within a target region of a nucleic acid sequence, as well as kits for use in the method. The method is particularly suitable for the detection of multiple polymorphisms or allelic variations and so may be used in diagnostic methods.

### BACKGROUND

Known fluorescence polymerase chain reaction (PCR) monitoring techniques include both strand-specific and generic DNA intercalator techniques that can be used on a few second-generation PCR thermal cycling devices. These reactions are carried out homogeneously in a closed tube format on these thermal cyclers. Reactions are monitored using a fluorimeter. The precise form of the assays varies but often relies on fluorescence energy transfer or FET between two fluorescent moieties within the system in order to generate a signal indicative of the presence of the product of amplification.

Generic methods utilise DNA intercalating dyes that exhibit increased fluorescence when bound to double stranded DNA species. Fluorescence increase due to a rise in the bulk concentration of DNA during amplifications can be used to measure reaction progress and to determine the target molecule copy number. Furthermore, by monitoring fluorescence with a controlled change of temperature, DNA melting curves can be generated, for example, at the end of PCR thermal cycling.

When generic DNA methods are used to monitor the rise in bulk concentration of nucleic acids, these processes can be monitored with a minimal time penalty (compared to some other known assays discussed below). A single fluorescent reading can be taken at the same point in every reaction. End point melting curve analysis can be used to discriminate artefacts from amplicon, as well as to discriminate amplicon strands. Melting peaks of products can be determined for concentrations that cannot be visualised by agarose gel electrophoresis.

In order to obtain high resolution melting data, for example for multiple samples, the melt experiment must be performed slowly on existing hardware, taking up to five minutes. However, by continually monitoring fluorescence amplification, a 3D image of the hysteresis of melting and hybridisation can be produced. This 3D image is amplicon-dependent and may provide enough information for product discrimination. However, the generic intercalator methods are only quasi-strand-specific and are therefore not very useful where strand-specific detection is required.

Strand-specific methods utilise additional nucleic acid reaction components to monitor the progress of amplification reactions. These methods often use fluorescence energy transfer (FET) as the basis of detection. One or more nucleic acid probes are labelled with fluorescent molecules, one of which is able to act as an energy donor and the other of which is an energy acceptor molecule. These are sometimes known as a reporter molecule and a quencher molecule respectively. The donor molecule is excited with a specific wavelength of light which falls within its excitation spectrum and, subsequently, it will emit light within its fluorescence emission wavelength. The acceptor molecule is also excited at this wavelength by accepting energy from the donor molecule by a variety of distance-dependent energy transfer mechanisms. A specific example of fluorescence energy transfer which can occur is Fluorescence Resonance Energy Transfer or “FRET”. Generally, the acceptor molecule accepts the emission energy of the donor molecule when they are in close proximity (e.g., on the same, or a neighbouring molecule). The basis of fluorescence energy transfer detection is to monitor the changes at donor and acceptor emission wavelengths.

Examples of molecules used as donor and/or acceptor molecules in FRET systems include, amongst others, SYBRGold, SYBRGreenI, Fluorescein, rhodamine, Cy5, Cy5.5 and ethidium bromide, as well as others such as SYTO dyes as listed, for example, in WO2007/093816.

There are two commonly used types of FET or FRET probes, those using hydrolysis of nucleic acid probes to separate donor from acceptor and those using hybridisation to alter the spatial relationship of donor and acceptor molecules.

Hydrolysis probes are commercially available as TaqMan™ probes. These consist of DNA oligonucleotides that are labelled with donor and acceptor molecules. The probes are designed to bind to a specific region on one strand of a PCR product. Following annealing of the PCR primer to this strand, Taq enzyme extends the DNA with 5′ to 3′ polymerase activity. Taq enzyme also exhibits 5′ to 3′ exonuclease activity. TaqMan™ probes are protected at the 3′ end by phosphorylation (or other blocking moiety) to prevent them from priming Taq extension. If the TaqMan™ probe is hybridised to the product strand, an extending Taq molecule may also hydrolyse the probe, liberating the donor from acceptor as the basis of detection. The signal in this instance is cumulative, the concentration of free donor and acceptor molecules increasing with each cycle of the amplification reaction.

If hydrolysis probes are used to detect the presence of a polymorphism within a target sequence, two separate probes are required, one which will hybridise to the sequence only when the polymorphism is present and one which will hybridise to the sequence only when the polymorphism is not present. Each probe must be labelled with a different donor/acceptor pair so that a change in fluorescence on hydrolysis can be detected separately for sequences where the polymorphism is present and those where it is absent. Therefore, a pair of separately labelled probes is required for each single polymorphism to be detected, using a total of four different fluorescent labels. This increases the cost of the overall detection system, particularly where more than one polymorphism is to be sought within a sequence. The number of probes required is further increased if more than one target sequence is to be detected in a sample.

In addition, the fact that signal generation is dependent upon the occurrence of probe hydrolysis reactions means that there is a time penalty associated with this method since the 5′-3′ hydrolysis process of the enzyme is much slower that the 5′-3′ polymerase activity. Furthermore, the presence of the probe may interrupt the smooth operation of the PCR process as it may “clamp” extension at high concentration. A further disadvantage is that it has been found that hydrolysis can become non-specific, particularly where large numbers of amplification cycles, for instance more than 50 cycles, are required. In these cases, non-specific hydrolysis of the probe will result in an unduly elevated signal.

This means that such techniques are not very compatible with rapid PCR methods which are now more prominent with the development of rapid hot air thermal cyclers such as the RapidCycler™ and LightCycler™ from Idaho Technologies Inc. Other rapid PCR devices are described, for example, in WO98/24548. The merits of rapid cycling over conventional thermal cycling have been reported elsewhere. Such techniques are particularly useful for example in detection systems for biological warfare where speed of result is important if loss of life or serious injury is to be avoided.

Furthermore, hydrolysis probes do not provide significant information with regard to hysteresis of melting since signal generation is, by and large, dependent upon hydrolysis of the probe rather than the melt temperature of the amplicon strand.

Hybridisation probes, an alternative to hydrolysis probes, are available in a number of forms. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated so FRET does not occur, forming the basis of detection.

Pairs of labelled oligonucleotides may also be used. As shown in FIG. 1A below, these hybridise in close proximity to one another on a PCR product strand bringing donor and acceptor molecules (e.g., fluorescein and rhodamine) together so that FRET can occur, as disclosed in WO97/46714, for example. Enhanced FRET is the basis of detection. The use of two probes requires the presence of a reasonably long known sequence so that two probes which are long enough to bind specifically can bind in close proximity to each other. This can be a problem in some diagnostic applications, where the length of conserved sequences in an organism which can be used to design an effective probe, such as the HIV virus, may be relatively short.

Furthermore, the use of pairs of probes involves more complex experimental design. For example, a signal provided by the melt of a probe is a function of the melting-off of both probes. Therefore, two separately labelled probes are required for the detection of each single sequence.

A variation of this type of system is shown in FIG. 1B and uses a labelled amplification primer with a single adjacent probe, also as disclosed in WO97/46714. However, such a system can only be used to detect a single target sequence which is relatively close to the site of the binding of the amplification primer, since the label on the probe and the label on the primer must be in sufficient proximity when the probe is bound for FRET to occur. For more than one target sequence to be detected, a separate amplification reaction must be carried out for each sequence.

A problem with this system is that, if equal amounts of forward and reverse amplicon strands are present in the amplification vessel, they will tend to preferentially hybridise to one another, out-competing probe/target sequence hybridisation during the signal phase of the reaction and causing the signal to “hook”. To overcome this, an amplification bias for the amplicon strand complimentary to the probe is introduced by inclusion of a significantly higher concentration of one amplification primer compared to the other amplification primer, as described in Bernard et al. (1998) Analyt. Biochem. 255 101-107.

WO 99/28500 describes a successful assay for detecting the presence of a target nucleic acid sequence in a sample, designed to overcome some of the problems with detection of multiple sequences using the above methods, such as the number of fluorescence labels and the number of labelled probes required. In this method, a DNA duplex binding agent and a probe specific for said target sequence, is added to the sample. The probe comprises a reactive molecule able to absorb fluorescence from or donate fluorescent energy to said DNA duplex binding agent. This mixture is then subjected to an amplification reaction in which target nucleic acid is amplified, conditions being induced either during or after the amplification process in which the probe hybridises to the target sequence. Fluorescence from said sample is monitored.

As the probe hybridises to the target sequence, a DNA duplex binding agent such as an intercalating dye is trapped between the strands. In general, this would increase the fluorescence at the wavelength associated with the dye. However, where the reactive molecule is able to absorb fluorescence from the dye (i.e., it is an acceptor molecule), it accepts emission energy from the dye by means of FET, especially FRET, and so it emits fluorescence at its characteristic wavelength. An increase in fluorescence from the acceptor molecule, which is of a different wavelength to that of the dye, will indicate binding of the probe in duplex form.

Similarly, where the reactive molecule is able to donate fluorescence to the dye (i.e., it is a donor molecule), the emission from the donor molecule is reduced as a result of FRET and this reduction may be detected. Fluorescence of the dye is increased more than would be expected under these circumstances.

The signal from the reactive molecule on the probe is a strand specific signal, indicative of the presence of specific target within the sample. Thus, the changes in fluorescence signal from the reactive molecule, which are indicative of the formation or destabilisation of duplexes involving the probe, are preferably monitored.

DNA duplex binding agents, which may be used in the process, are any entity which adheres or associates itself with DNA in duplex form and which is capable of acting as an energy donor or acceptor. Particular examples are intercalating dyes as are well known in the art.

The use of a DNA duplex binding agent such as an intercalating dye and a probe which is singly labelled has advantages in that these components are much more economical than other assays in which doubly labelled probes are required. By using only one probe, the length of known sequence necessary to form the basis of the probe can be relatively short and therefore the method can be used, even in difficult diagnostic situations. The assay in this case is known as ResonSense®. However, this method is still limited in that different sequences which are located close together on the same DNA molecule are difficult to detect, as the result of the constraints in availability of space for the different pairs of probes to bind.

WO02/097132 describes a variation of the ResonSense® method in which a particular probe type is utilised. WO2004/033726 describes a further variation in which a DNA duplex binding agent which can absorb fluorescent energy from the fluorescent label on the probe but which does not emit visible light is used, so as to avoid interfering with the signal. WO2007/093816 describes a particularly useful dye label system.

### SUMMARY OF INVENTION

According to a first aspect of the invention, there is provided a method of detecting the presence in a sample of a first target sequence and a second target sequence within a test region of a nucleic acid sequence, comprising conducting a nucleic acid amplification reaction to form a forward amplicon strand and a reverse amplicon strand of the test region, contacting the forward amplicon strand with a first probe labelled with a first FRET label and capable of hybridising to the first target sequence or complement thereof in the forward amplicon strand, and contacting the reverse amplicon strand with a second probe labelled with a second FRET label and capable of hybridising to the second target sequence or complement thereof in the reverse amplicon strand; wherein the nucleic acid amplification reaction is conducted using a forward amplification primer labelled with a third FRET label and a reverse amplification primer labelled with a fourth FRET label, the forward primer being incorporated into the forward amplicon strand and the second primer being incorporated into the reverse amplicon strand during the amplification reaction; and further wherein the first and third FRET labels form a first FRET pair and the second and fourth FRET labels form a second FRET pair, each FRET pair comprising a donor label; the method further comprising the steps of exposing the sample to an excitation source having a wavelength which excites the donor label in the first FRET pair and the donor label in the second FRET pair, detecting fluorescence from the sample and relating this to the presence or absence of the first and second target sequences.

Advantageously, this enables the detection of more than one target sequence located closely together, within a target region of nucleic acid, which is not possible with dual probe techniques which require binding of both probes to the same amplicon strand. As mentioned above, detection of two separate sequences within a test region using dual probes requires space on the amplicon strand for binding of a total of four probes. In an advantage over probe hydrolysis techniques, the method of the invention enables detection of a polymorphism within more than target sequence using only a single labelled probe for each target sequence, rather than two probes, corresponding to the target sequence with the polymorphism present or absent, labelled with a total of four different fluorescence labels.

The fact that the excitation source (which may be any typical source having emissions in the electromagnetic spectrum, for example, in the visible range of the electromagnetic spectrum) has a wavelength which excites both the donor labels present in the system has the advantage that the method can be carried out using an instrument comprising only a single excitation source such as an LED. Prior art methods, such as that disclosed in WO2007/018734, required excitation of each donor label at a different wavelength. In a preferred embodiment of the present invention, both donor labels are the same as one another, i.e., the donor labels of the FRET pairs are identical. Use of a single LED simplifies the optical arrangement of the fluorimeter. Ultimately this reduces the overall cost of the instrumentation required to implement this chemistry.

Amplification is suitably effected using known amplification reactions such as the polymerase chain reaction (PCR), strand displacement assay (SDA), transcriptional mediated amplification (TMA) or NASBA, but preferably PCR. The nucleic acid polymerase is preferably a thermostable polymerase such as Taq polymerase. Suitable conditions under which the amplification reaction can be carried out are well known in the art. The optimum conditions may be variable in each case depending upon the particular amplicon strand involved, the nature of the primers used and the enzymes employed. The optimum conditions may be determined in each case by the skilled person. Typical denaturation temperatures are of the order of 95° C., typical annealing temperatures are of the order of 55° C. and extension temperatures are of the order of 72° C.

Suitable FRET labels are a donor molecule such as a fluorescein (or derivatives) and an acceptor molecule such as a rhodamine dye or another dye such as cyanine dyes, for example Cy5. These may be attached to the primers and probes in a conventional manner. In order for FRET to occur between the label on the primers and probes, the fluorescent emission of the label which acts as the donor must be of a shorter wavelength than the element acceptor.

Preferably, the molecules forming FRET pairs and used as labels produce sharp Gaussian peaks, so that there is little or no overlap in the wavelengths of the emission. Under these circumstances, it may not be necessary to resolve the signal produced by the donor label, with a simple measurement of the acceptor signal being sufficient. However, where there is a spectral overlap in the fluorescent signals from the donor and acceptor molecules, this can be accounted for in the results. Therefore, preferably, the fluorescence of both the donor and the acceptor molecule are monitored and the relationship between the emissions calculated.

The method may additionally comprise the step of determining a melting profile of the first probe/forward amplicon strand hybrid by monitoring fluorescence from the sample at a first wavelength. Therefore, the presence of a polymorphism in the first target sequence is detectable by detection of a different peak melting temperature (e.g., a lower temperature) of the forward amplicon strand compared to the peak melting temperature in a sample not having the polymorphism. As shown in FIG. 3A, the probe binds to the target sequence across the point of the possible polymorphism, the site of the polymorphism being indicated by χ. The binding of the probe to the target sequence is less stringent when the polymorphism is present, since the probe is complementary to the wild-type sequence. Therefore, the melting temperature for the probe/target hybrid is lower when the polymorphism is present than the melting temperature for the hybrid when the polymorphism is absent, as shown in FIG. 3B.

Furthermore, the method may alternatively or additionally comprise the step of determining a melting profile of the second probe/reverse amplicon strand hybrid by monitoring fluorescence from the sample at a second wavelength, so that the presence of a polymorphism in the second target sequence is detected by detection of a different peak melting temperature (e.g., a lower temperature) of the reverse amplicon strand compared to the peak melting temperature in a sample not having the polymorphism.

Therefore, a polymorphism may be detected in both target sequences by analysing the fluorescence of a single amplification reaction sample at only two wavelengths. Prior art methods would either require more than one amplification reaction to be carried out, or would require detection of fluorescence from the binding of more than one probe to each target region, depending on whether or not each polymorphism were present. Furthermore, excitation of both donor labels in the method is at a single wavelength, preferably by a single excitation source. Some earlier methods required the use of several excitation wavelengths and, therefore, several excitation sources.

Since the system utilises the labelling of both the forward and reverse primers, more than one sequence and/or polymorphism can be detected within a test region to be amplified by the primers. This is achieved by use of more than one probe each labelled so as to be able to form a different FRET relationship with one of the labelled primers to the relationship formed by other probes and arranged to be complementary to sequences in either the forward or reverse amplicon strand.

In a further embodiment, the melting profile of the first probe/forward amplicon strand hybrid and the profile of the second probe/reverse amplicon strand hybrid may be detectable at the same wavelength. This is possible when the melting peak of the different hybrids is different in each of the situations in which: (a) both target sequences are wild-type; (b) one of the target sequences includes a polymorphism; and (c) both target sequences include a polymorphism. The presence or absence of each target sequence and the presence or absence of each polymorphism may then be determined by comparison with the melt profile of known sequences. This provides an additional advantage that only two different FRET labels may be required, one to be used as the first and second FRET labels and one to be used as the third and fourth FRET labels. This reduces the overall cost of the assay and increases the multiplexing capacity of the assay, since the non-utilised dye may be used to address some other investigation.

In one embodiment, the first FRET label is a fluorescence donor molecule and the third FRET label is a fluorescence acceptor molecule able to absorb fluorescence from the first FRET label, or the third FRET label is a fluorescence donor molecule and the first FRET label is a fluorescence acceptor molecule able to absorb fluorescence from the third FRET label. Such a donor/acceptor relationship between the two FRET labels is defined herein as a “FRET pair”.

Alternatively or additionally the second FRET label is a fluorescence donor molecule and the fourth FRET label is a fluorescence acceptor molecule able to absorb fluorescence from the second FRET label, or the fourth FRET label is a fluorescence donor molecule and the second FRET label is a fluorescence acceptor molecule able to absorb fluorescence from the fourth FRET label.

In some embodiments, the first and second FRET labels may be different from one another, and the third and fourth FRET labels the same as one another, or the first and second FRET labels may be the same as one another and the third and fourth FRET labels different from one another. Alternatively, the first and second FRET labels may be the same as one another and the third and fourth FRET labels may be the same as one another. In any embodiment, two separate FRET relationships are provided, one between the pair of labels on the first probe and forward primer and one between the pair of labels on the second probe and reverse primer. The donor labels are selected to be excitable at the same wavelength and may, for example, be the same label. Each of the FRET relationships may be separately detectable by monitoring the fluorescence of the amplification reaction sample at different wavelengths, according to the emission spectra of the acceptor molecules in each FRET pair. Alternatively, as discussed above, the different FRET relationships may be detectable at a single wavelength and distinguishable by the melting profile of each probe/amplicon strand hybrid.

For example, the first FRET label may be IDT TYE665, the second FRET label may be IDT TYE705 (both available from Integrated DNA Technologies BVBA, Belgium) and the third and fourth FRET labels may both be a donor molecule such as a fluorescein, for example, a FAM isomer. Where different melting peaks result for each probe/amplicon strand hybrid, both the first and second FRET labels may be TYE665, or both may be TYE705, with the third and fourth FRET labels being a fluorescein. An alternative to TYE665 is Cy5; an alternative to TYE705 is Cy5.5. The skilled person can readily determine suitable FRET labels to be used as each of the first, second, third and fourth FRET labels.

Surprisingly, using the method of the invention, inhibition of probe/amplicon strand hybrid formation as the result of the hybridisation of the amplicon strands to one another is not observed. Therefore, biased amplification of one or the other amplicon strands, to enable probe hybridisation, advantageously is not required. This is unexpected in view of the disclosures of WO97/46714 and Bernard et al. (1998).

In the method of the invention, the sample may be subjected to conditions under which the probe hybridises to the samples either during and/or after the amplification reaction has been completed. The process allows the detection to be effected in a homogenous manner, in that the amplification and monitoring can be carried out in a single container with all reagents added initially. No subsequent reagent addition steps are required. Neither is there any need to affect the method in the presence of solid supports (although this is an option as discussed further below).

For example, where the probes are present throughout the amplification reaction, the fluorescent signal may allow the progress of the amplification reaction to be monitored. This may provide a means for quantification of the amount of the target sequences present in the sample.

The probes may comprise a nucleic acid molecule such as DNA or RNA, which will hybridise to the target nucleic acid sequences when these are in single stranded form. In this instance, the method will involve the use of conditions which render the target nucleic acid sequences single stranded. Alternatively, the probes may comprise a molecule such as a peptide nucleic acid which may specifically bind the target sequences in double stranded form.

In particular, the amplification reaction used will involve a step of subjecting the sample to conditions under which any of the target nucleic acid sequence present in the sample becomes single stranded, such as PCR or SDA. It is possible then for the probe to hybridise during the course of the amplification reaction provided appropriate hybridisation conditions are encountered.

In a preferred embodiment, the probe may be designed such that these conditions are met during each cycle of the amplification reaction. Thus, at some point during each cycle of the amplification reaction, the probe will hybridise to the target sequence in the amplicon strand and a signal will be generated as a result of the FRET, given the proximity of the probe to the labelled primer incorporated into the amplicon strand. As the amplification proceeds, the probe will be separated or melted from the amplicon strand which incorporates the labelled primer and so the signal generated from the FRET pair will change.

By monitoring the fluorescence of the label(s) from the sample during each cycle, the progress of the amplification reaction can be monitored in various ways. For example, the data provided by melting peaks can be analysed, for example by calculating the area under the melting peaks and this data plotted against the number of cycles.

Fluorescence is suitably monitored using a known fluorimeter. The signals from these, for instance in the form of photo-multiplier voltages, are sent to a data processor board and converted into a spectrum associated with each sample tube. Multiple tubes, for example 96 tubes, can be assessed at the same time. Data may be collected in this way at frequent intervals, for example once every 10 ms, throughout the reaction.

The spectra generated in this way can be resolved, for example, using “fits” of pre-selected fluorescent moieties such as dyes, to form peaks representative of each signalling moiety (i.e., the FRET labels). The areas under the peaks can be determined which represents the intensity value for each signal and, if required, expressed as quotients of each other. The differential of signal intensities and/or ratios will allow changes in FRET to be recorded through the reaction or at different reaction conditions, such as temperatures. The integral of the area under the differential peaks (with respect to temperature) will allow intensity values for the FET or FRET effects to be calculated.

This data provides one means to quantitate the amount of target nucleic acid present in the sample.

Each probe may either be free in solution or immobilised on a solid support, for example to the surface of a bead such as a magnetic bead, useful in separating products, or the surface of a detector device, such as the waveguide of a surface plasmon resonance detector. The selection will depend upon the nature of the particular assay being looked at and the particular detection means being employed.

In order to achieve a fully reversible signal which is directly related to the amount of amplification product present at each stage of the reaction and/or where speed of reaction is of the greatest importance, for example in rapid PCR, it is preferable that each probe is designed such that it is released intact from the target sequence and so may take part again in the reaction. This may be, for example, during the extension phase of the amplification reaction. However, since the signal is not dependent upon probe hydrolysis, the probe may be designed to hybridise and melt from the target sequence at any stage during the amplification cycle, including the annealing or denaturing phase of the reaction. Such probes will ensure that interference with the amplification reaction is minimised.

Where probes which bind during the extension phase are used, their release intact may be achieved by using a 5′-3′ exonuclease-lacking enzyme such as Stoffle fragment of Tag or Pwo. This may be useful when rapid PCR is required, as hydrolysis steps are avoided.

When used in this way, it is important to ensure that the probes are not extended during the extension phase of the reaction. Therefore, the 3′ end of each probe is blocked, for example, by incorporation of the fluorophore at the 3′ end and/or by the inclusion of a 3′ blocking moiety such as phosphate.

The data generated in this way can be interpreted in various ways. In its simplest form, an increase in fluorescence of the acceptor molecule in the course of or at the end of the amplification reaction is indicative of an increase in the amount of the target sequence present, suggestive of the fact that the amplification reaction has proceeded and therefore the target sequence was in fact present in the sample. However, as outlined above, quantification is also possible by monitoring the amplification reaction throughout. Finally, again as mentioned above, it is possible to obtain characterisation data and in particular melting point analysis, either as an end point measure or throughout, in order to obtain information about the sequence.

In some embodiments, probe hybridisation may occur at a temperature lower than the temperatures used in the amplification reaction. This advantageously increases the options available when designing suitable probes for use in detecting particular target sequences in a sample.

In one embodiment, the nucleic acid sequence in the sample, comprising the test region, is RNA and the method comprises a step of carrying out a reverse transcription reaction. For example, detection of multiple polymorphisms within the sequence of the virus Influenza A is required to determine whether the virus is resistant to the antiviral drug Tamiflu®. Resistance to this drug has been found to be present when the polymorphisms causing the amino acid changes H274Y and N294S are present in the neuraminidase gene in N1 subtypes of Influenza A. The two polymorphisms are located within 60 nucleic acids of each other and are not detectable (or not rapidly and economically detectable) using the techniques of the prior art in a single amplification assay system, as the result of constraints in multiple probe binding in dual hybridisation probe systems, or the need for a large number of differently labelled probes in a probe hydrolysis system. These problems have been solved by providing the method according to the invention.

According to the second aspect of the invention, there is provided a kit comprising a first nucleic acid probe labelled with a first FRET label, a second nucleic acid probe labelled with a second FRET label, a forward nucleic acid amplification primer labelled with a third FRET label and a reverse nucleic acid amplification primer labelled with a fourth FRET label, wherein the first and third FRET labels form a first FRET pair including a first donor label and the second and fourth FRET labels form a second FRET pair including a second donor label, the first and second donor labels being excitable at the same wavelength. The first and second donor labels may be the same. The kit may be for use in a method according to the first aspect of the invention.

For example, the first FRET label may be IDT TYE665, the second FRET label may be IDT TYE705 and the third and fourth FRET labels may both be a fluorescein such as a FAM isomer. In some embodiments, both the first and second FRET labels may be TYE665, or both may be TYE705. An alternative to TYE665 is Cy5 and an alternative to TYE705 is Cy5.5.

In some embodiments, the kit may further comprise a DNA polymerase such as a DNA-dependent DNA polymerase (e.g., Taq, Pwo) or a RNA- or DNA-dependent DNA polymerase (e.g., Tth). The kit may comprise more than one DNA polymerase of any type.

In an embodiment of this aspect of the invention, the first FRET label is a fluorescence donor molecule and the third FRET label is a fluorescence acceptor molecule able to absorb fluorescence from the first FRET label, or the third FRET label is a fluorescence donor molecule and the first FRET label is a fluorescence acceptor molecule able to absorb fluorescence from the third FRET label. Alternatively or additionally, the second FRET label is a fluorescence donor molecule and the fourth FRET label is a fluorescence acceptor molecule able to absorb fluorescence from the second FRET label, or the fourth FRET label is a fluorescence donor molecule and the second FRET label is a fluorescence acceptor molecule able to absorb fluorescence from the fourth FRET label.

The first and second FRET labels may be different from one another, and the third and fourth FRET labels the same as one another, or the first and second FRET labels may be the same as one another and the third and fourth FRET labels different from one another. In some embodiments, as outlined above, the first and second FRET labels may be the same as one another and the third and fourth FRET labels may be the same as one another. In any embodiment, two separate FRET relationships are provided, one between the pair of labels on the first probe and forward primer and one between the pair of labels on the second probe and reverse primer. These may be distinguishable as the results of fluorescence detectable at different wavelengths, or the result of different melting peaks for different probe/amplicon strand hybrids. The donor labels are selected to be excitable at the same wavelength and may, for example, be the same label.

The kit may further comprise a reverse transcriptase, i.e., an RNA-dependent DNA polymerase (e.g., from Moloney-Murine Leukemia Virus—MMULV—or Avian Myeloblastosis Virus—AMV).

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith. Moreover, unless stated otherwise, any feature disclosed herein may be replaced by an alternative feature serving the same or a similar purpose.

### BRIEF DESCRIPTION OF THE FIGURES

FIG. 1A is a diagram showing a prior art technique to detect a target nucleotide sequence using two probes each labelled with one half of a FRET pair; and

FIG. 1B is a diagram showing a prior art technique to detect a target nucleotide sequence using a primer and a probe, each labelled with one half of a FRET pair.

Embodiments of the invention will now be described, by way of example only, with reference to the following FIGS. 2-5 in which:

FIG. 2 shows the method according to the invention to detect two target sequences within a test region of a nucleic acid sequence using two probes but a single set of amplification primers;

FIG. 3 shows the principle used to detect the presence or absence of a polymorphism χ at a given locus, with 3A showing the location of the polymorphism relative to the probe binding and 3B showing the melt analysis from a sample where both the wild-type and polymorphism sequence are present in the sample;

FIG. 4 shows the results of screening for the polymorphism causing the H274Y mutation by detection of fluorescence at 670 nm using a target consensus cDNA sequence developed by comparison of all known H5N1 viruses and primer/probe set 2 (below), with the panels showing the melt analysis for A: a non-template control, B: a sample containing a wild-type cDNA template, C: a sample containing a cDNA template including the N294S mutation, D: a sample containing a cDNA template including the H274Y mutation, E: a sample containing a cDNA template including both the N294S and H274Y mutations and F: the melt analysis for all samples tested; the dotted line shows the melting peak for the presence of the H274Y polymorphism and the dashed line shows the melting peak for the wild-type sequence at the same locus; and

FIG. 5 shows the results of screening for the polymorphism causing the N294S mutation by detection of fluorescence at 705 nm using a target consensus cDNA sequence developed by comparison of all known H5N1 viruses and primer/probe set 2, with the panels showing the melt analysis for A: a non-template control, B: a sample containing a wild-type cDNA template, C: a sample containing a cDNA template including the N294S mutation, D: a sample containing a cDNA template including the H274Y mutation, E: a sample containing a cDNA template including both the N294S and H274Y mutation and F: the melt analysis for all samples tested; the dotted line shows the melting peak for the presence of the N294S polymorphism and the dashed line shows the melting peak for the wild-type sequence at the same locus.

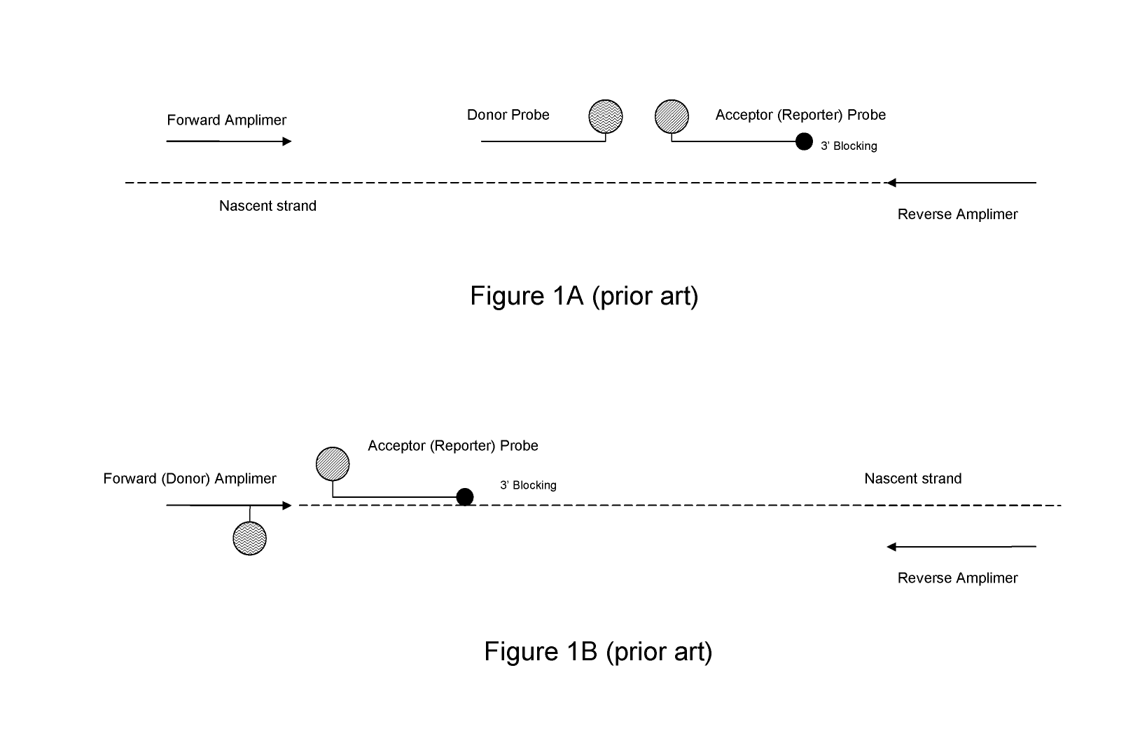

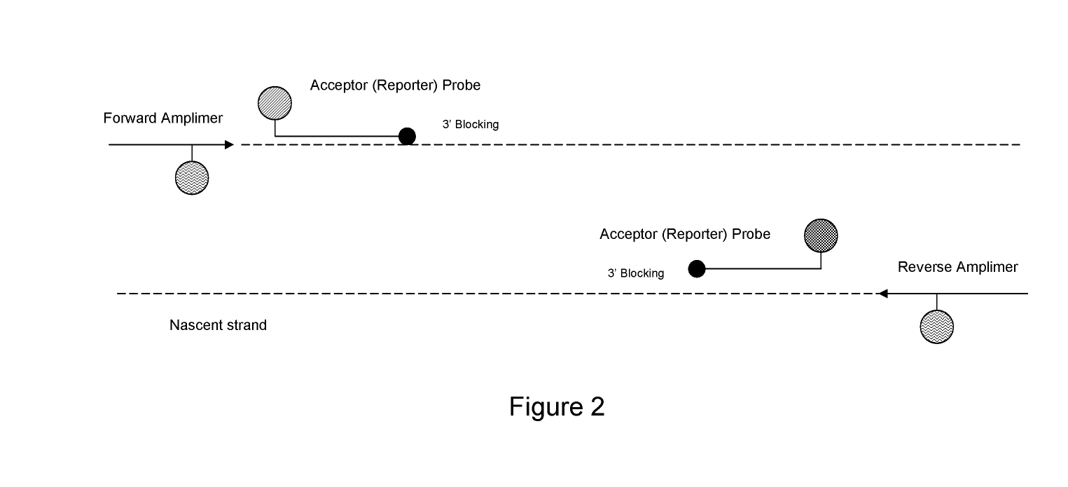

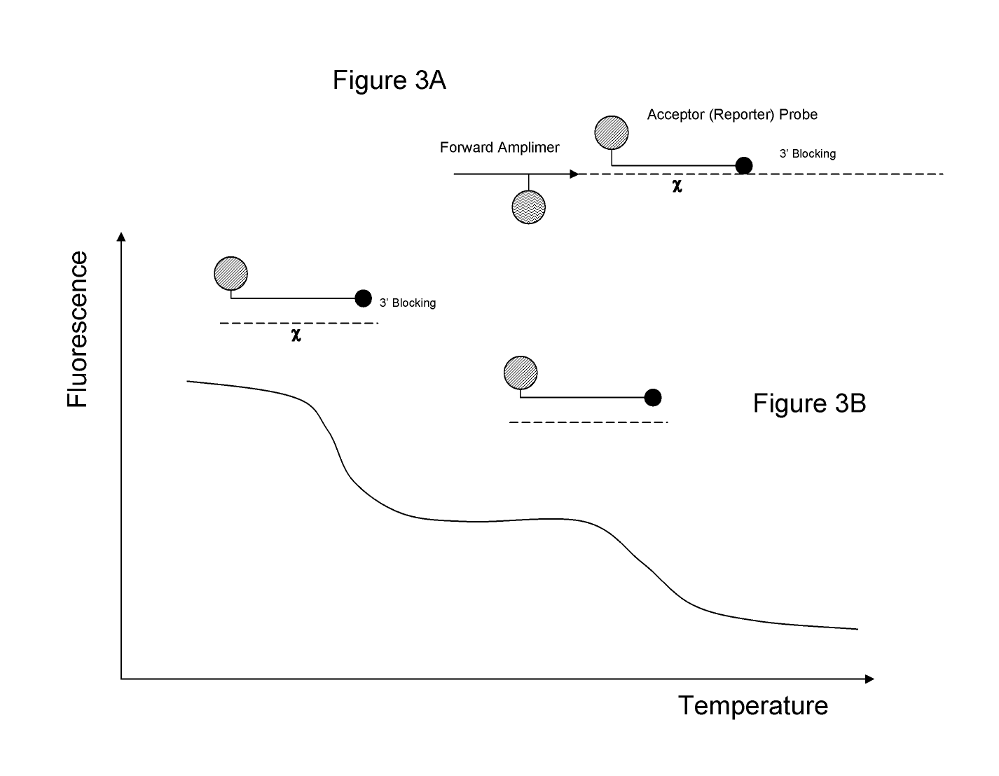

### EXAMPLES

An example of a situation where detection of several target sequences located closely together within a single nucleic acid would be useful is in the field of influenza diagnosis. Influenza viruses are RNA viruses and the most common type of flu virus is Influenza A. Within Influenza A there are several serotypes categorised on the basis of antibody responses to them, of which the most well known are H5N1 (avian flu) and H1N1 (swine flu). The “H” denotes hemagglutinin and the “N” neuraminidase, both proteins expressed on the surface of the flu virus and which exhibit the variations which give rise to the different antibody responses to the different serotypes of the virus.

During the 2009 worldwide outbreak of H1N1 swine flu, the antiviral drug Tamiflu® was a key means of suppressing viral infection and therefore limiting the spread of the virus. However, some strains of the virus were found to be resistant to Tamiflu® but identification of individuals carrying such a strain was only possible when treatment with Tamiflu® had been found to be ineffective, at which stage alternative treatment using a drug such as Relenza® would be appropriate. However, it would have been preferable to be able to identify the presence of a resistant strain before treatment began, so as to provide effective treatment more quickly and also to reduce the risk of the further transmission of the Tamiflu® resistant strain.

Resistance to the Tamiflu® drug is most commonly present when the polymorphisms causing the amino acid changes H274Y and N294S are present in the neuraminidase gene in N1 subtypes of Influenza A. A screening method to identify the presence of these polymorphisms is therefore required, which can provide rapid results at a reasonable cost. In approaching this, the inventor found that methods utilising probe hydrolysis would require multiple nucleic acid amplification reactions with multiple labelled probes to be carried out, to enable detection of the presence of polymorphisms in two sequences. This would result in a slow and costly assay system. In addition, the close location of the polymorphisms would require generation of overlapping amplicon strands, which would tend to form a heteroduplex and/or to co-migrate on a gel. These problems also applied to methods utilising hybridisation probe methods, with additional drawbacks resulting from the close proximity of the two polymorphisms to be detected, within the same nucleic acid sequence.

In response to these problems, the method of the present invention was devised and is exemplified below.

The following cDNA sequence corresponds to a consensus sequence for a portion of the RNA sequence from all known strains of H5N1 influenza viruses. This part of the sequence includes the codons which, when altered, result in the H274Y and N294S mutations in the neuraminidase protein:

(SEQ ID NO: 1)
AAAGGGAAAGTGGTTAAATCAGTCGAATTGGATGCTCCTAATTATCACT
ATGAGGAGTGCTCCTGTTATCCTTTTGATGCCGGCGAAATCACATGTGT
GTGCAGGGATAATTGGCATGGCTCAAATAGGCCATGGGTATCTTTCAAT
CAAAATT

The underlined codon “CAC” is that encoding the amino acid Histamine at position 274 in the neuraminidase protein. Alteration of this to TAT or TAC results in expression of Tyrosine at this position. The underlined codon “AAT” is that encoding the amino acid Asparagine at position 294. Alteration of this from AAT to TCT, TCC, TCA or TCG results in expression of Serine at this position.

The following sets of primers and hybridisation probes were identified, by methods routinely used by the skilled person (use of the open-source software JALVIEW in combination with the EMBL search toolset), as being suitable for amplification of regions of SEQ ID NO:1 which encompassed the two polymorphism sites. Probes for the N294S polymorphism were developed so as to be complementary to the reverse amplicon strand.

TABLE 1
Primer/Probe Set 1 (H5N1)

| | | | | SEQ |
|---|---|---|---|---|
| Primer/ | | | FRET | ID |
| probe | Name | Sequence 5′-3′ | label | NO |
| Forward | TAMH5N1MLA_F1 | AGTCGAATTGGATG | Fluores- | 2 |
| primer | | CTCCTAAT | cein | |
| Reverse | TAMH5N1MLA_R1 | GCCTATTTGAGCCA | Fluores- | 3 |
| primer | | TGC | cein | |
| Probe | TAMMLH5N1A_H274Y | AGGAGCACTCCTCA | TYE665 | 4 |
| | | TAGTGATAATTAG | | |
| Probe | TAMMLH5N1A_N294S | GTGCAGGGATAATT | TYE705 | 5 |
| | | GGCATG | | |

TABLE 2
Primer/Probe Set 2 (H5N1)

| | | | | SEQ |
|---|---|---|---|---|
| Primer/ | | | FRET | ID |
| probe | Name | Sequence 5′-3′ | label | NO |
| Forward | TAMH5N1ML_F1 | AGTCGAATTGGATG | Fluores- | 6 |
| primer | | CTCCTA | cein | |
| Reverse | TAMH5N1ML_R1 | CCCATGGCCTATTT | Fluores- | 7 |
| primer | | GAGC | cein | |
| Probe | TAMMLH5N1_H274Y | GGATAACAGGAGCA | TYE665 | 8 |
| | | CTCCTCATAGTGA | | |
| | | TA | | |
| Probe | TAMMLH5N1_N294S | GTGTGTGCAGGGAT | TYE705 | 9 |
| | | AATTGGCA | | |

TABLE 3
Primer/Probe Set 3 (H5N1)

| | | | | SEQ |
|---|---|---|---|---|
| Primer/ | | | FRET | ID |
| probe | Name | Sequence 5′-3′ | label | NO |
| Forward | TAMH5N1ML_F2 | GAATTGGATGCTCC | Fluores- | 10 |
| primer | | TAATTATCACT | cein | |
| Reverse | TAMH5N1ML_R2 | CTATTTGAGCCATG | Fluores- | 11 |
| primer | | CCAATTA | cein | |
| Probe | TAMMLH5N1_H274Y | GGATAACAGGAGCA | TYE665 | 8 |
| | | CTCCTCATAGTGA | | |
| | | TA | | |
| Probe | TAMMLH5N1_N294S | GTGTGTGCAGGGAT | TYE705 | 9 |
| | | AATTGGCA | | |

TABLE 4
Primer/Probe Set 4 (H1N1)

| | | | | SEQ |
|---|---|---|---|---|
| Primer/ | | | FRET | ID |
| probe | Name | Sequence 5′-3′ | label | NO |
| Forward | TAMH1N1MLA_F1 | TAGAGTTGAATGCA | Fluores- | 12 |
| primer | | CCCAATT | cein | |
| Reverse | TAMH1N1MLA_R1 | AGGTCGATTTGAAC | Fluores- | 13 |
| primer | | CATGC | cein | |
| Probe | TAMH1N1MLA_H274Y | GGAACATTCCTCAT | TYE665 | 14 |
| | | AATGAAAATTGGG | | |
| | | TG | | |
| Probe | TAMH1N1MLA_N294S | CAGGGACAACTGG | ITYE705 | 15 |
| | | CATG | | |

TABLE 5
Primer/Probe Set 5 (H1N1)

| | | | | SEQ |
|---|---|---|---|---|
| Primer/ | | | FRET | ID |
| probe | Name | Sequence 5′-3′ | label | NO |
| Forward | TAMH1N1ML_F1 | CAATAGAGTTGAAT | Fluores- | 16 |
| primer | | GCACCCA | cein | |
| Reverse | TAMH1N1ML_R1 | CCAAGGTCGATTTG | Fluores- | 17 |
| primer | | AACCATG | cein | |
| Probe | TAMH1N1ML_H274Y | CTGGGTAACAGGAA | TYE665 | 18 |
| | | CATTCCTCATAATG | | |
| | | AAA | | |
| Probe | TAMH1N1ML_N294S | TGATGTGTGTATGC | TYE705 | 19 |
| | | AGGGACAACTG | | |

TABLE 6
Primer/Probe Set 6 (H1N1)

| | | | | SEQ |
|---|---|---|---|---|
| Primer/ | | | FRET | ID |
| probe | Name | Sequence 5′-3′ | label | NO |
| Forward | TAMH1N1ML_F2 | TTGAATGCACCCAA | Fluores- | 20 |
| primer | | TTTTCATT | cein | |
| Reverse | TAMH1N1ML_R2 | GATTTGAACCATGC | Fluores- | 21 |
| primer | TTG | CAG | cein | |
| Probe | TAMH1N1ML_H274Y | CTGGGTAACAGGAA | TYE665 | 18 |
| | | CATTCCTCATAATG | | |
| | | AAA | | |
| Probe | TAMH1N1ML_N294S | TGATGTGTGTATGC | TYE705 | 19 |
| | | AGGGACAACTG | | |

Polymerase chain reactions were carried out using the above sets of primers and probes and, at the conclusion of the PCR, a melt analysis was carried out for each sample.

The reagents used are set out in Table 7:

TABLE 7
components of PCR reaction mixtures

| | | | | | Volume per |
|---|---|---|---|---|---|
| | | | | | 20 μl Reaction |
| Reagent | Stock Conc | | Final Conc | | (μl) |
| Tris-HCl pH 8.8 | 500 | mM | 50 | mM | 2 |
| BSA | 20 | mg/ml | 0.25 | μg/μl | 0.25 |
| MgCl2 | 100 | mM | 3 | mM | 0.6 |
| dUTPs | 2 | mM | 0.2 | mM | 2 |
| Forward Primer | 10 | μM | 0.5 | μM | 1 |
| Reverse primer | 10 | μM | 0.5 | μM | 1 |
| TYE665-labelled probe | 10 | μM | 0.2 | μM | 0.4 |
| TYE705-labelled probe | 10 | μM | 0.2 | μM | 0.4 |
| Anti-Taq Polymerase | 5 | U/μl | 0.08 | U/μl | 0.32 |
| Antibody | | | | | |
| Taq Polymerase | 5 | U/μl | 0.04 | U/μl | 0.16 |

| | | | |
|---|---|---|---|
| Template plasmid | — | | 2 |
| Nuclease-free H2O | — | — | 9.87 |

Table 8 shows the PCR temperature cycling conditions:

TABLE 8
PCR and melt analysis conditions (LightCycler 2.0)

| Phase | | | | Target | Hold | Transition | Florescence | |
|---|---|---|---|---|---|---|---|---|
| | | No of | Segment | Temp | Time | Rate | Acquisition | |
| Number | Type | Cycles | Number | ° C. | s | ° C./s | Type | Channels |
| 1 | HOLD | 1 | 1 | 95 | 30 | 20 | — | — |
| 2 | Amplify | 50 | 1 | 95 | 5 | 20 | — | — |
| | | | 2 | 55 | 20 | 20 | Single | ALL |
| | | | 3 | 74 | 5 | 20 | — | — |
| 3 | MELT | 1 | 1 | 40 | 15 | 20 | — | — |
| | | | 2 | 95 | 0 | 0.1 | Continuous | ALL |

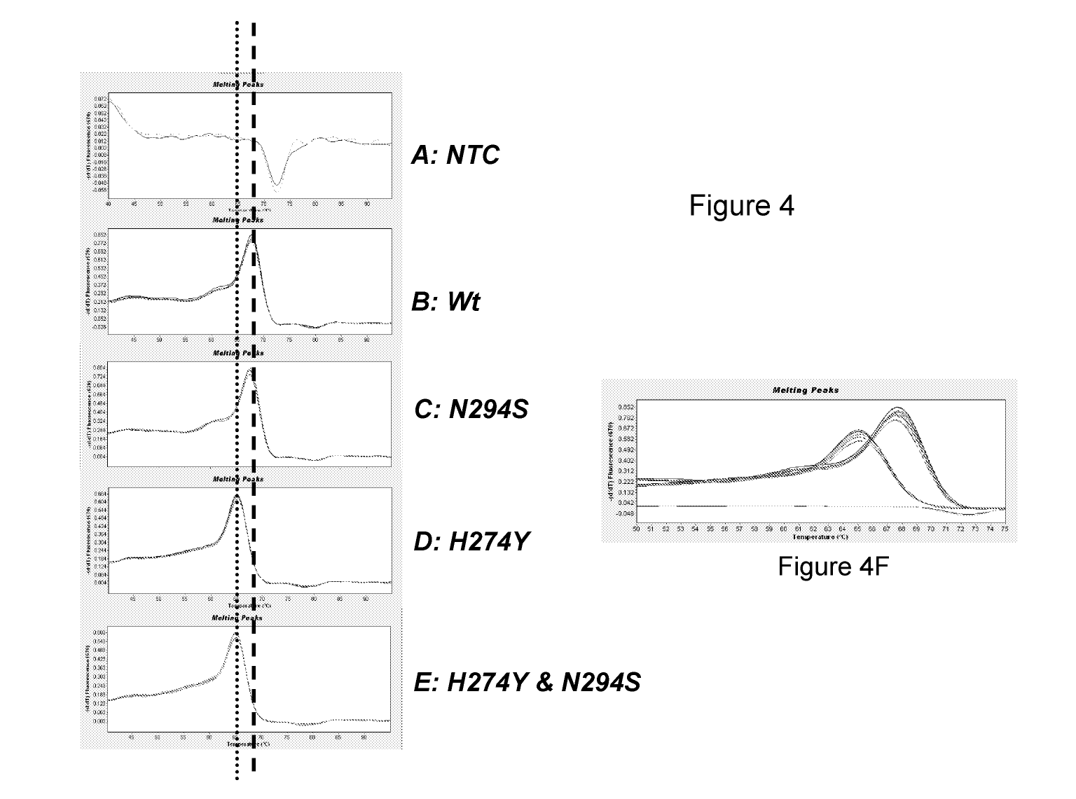

By way of example, the melt analysis results from an experiment conducted using primer/probe set 2 (Table 2) and detection of fluorescence at 670 nm are shown in FIG. 4 (in which panel A shows non-template control), to show detection of binding of the H274Y probe to the target. FIG. 4B shows the fluorescence peak for a wild type sample (marked with a dashed line), with FIG. 4D showing the lower temperature fluorescence peak (marked with a dotted line) for a sample having the H274Y polymorphism. FIG. 4C is a sample known to have the N294S polymorphism, showing that the temperature of the melting peak is as for wild type. However, a sample having both the N294S and H274Y polymorphisms has a melting peak corresponding to the H274Y sample (FIG. 4E). FIG. 4F shows the combined results from several samples.

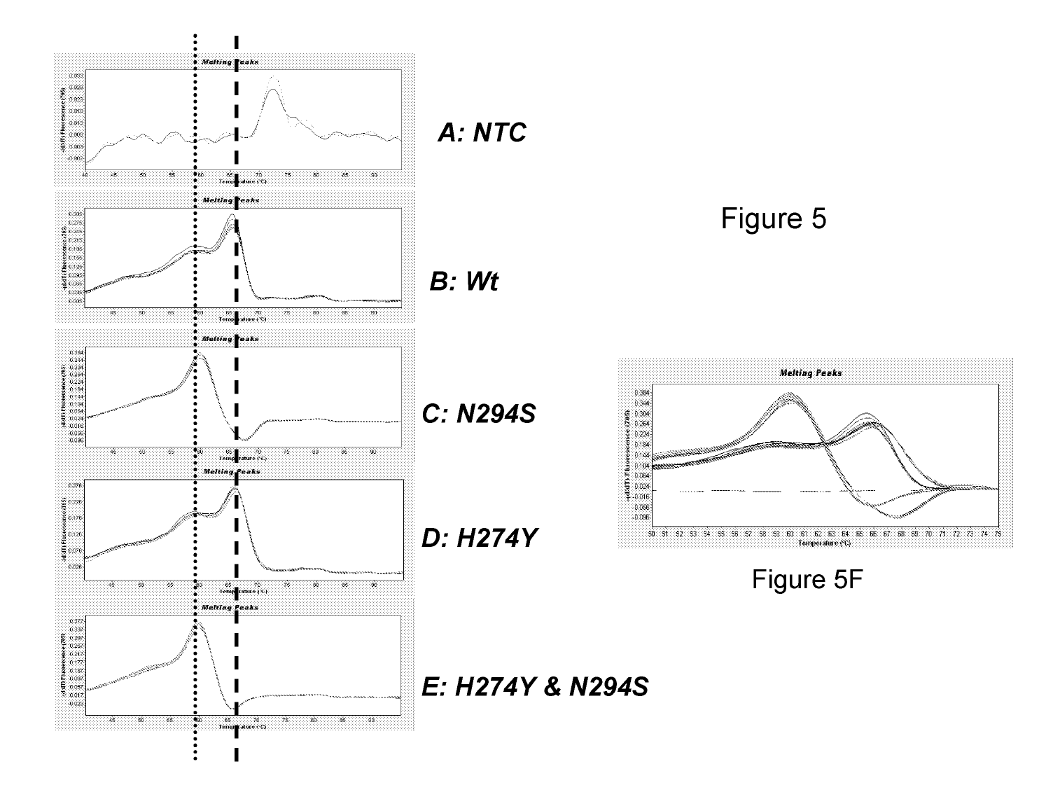

Again by way of example, the melt analysis results from the experiment conducted using primer/probe set 2 (Table 2) and detection of fluorescence at 705 nm are shown in FIG. 5, to show detection of binding of the N294S probe to the target. Again, the panels show that the presence of the N294S polymorphism results in a reduction in the temperature of the melting peak (dashed line is wild type, dotted line is N294S polymorphism). FIG. 5F shows the combined results of several samples, showing that the genotype of the flu strain in a particular sample can be distinguished using this method.

Therefore, as shown in FIGS. 5 and 6, by use of a single nucleic acid amplification reaction sample and by melt analysis of that single sample measuring fluorescence at just two wavelengths, the presence of two target sequences and the presence or absence of a polymorphism within each of these target sequences can be determined. This provides a rapid diagnostic assay for which the cost is kept down by use of only three different fluorescent labels in total.

1. Method of detecting the presence in a sample of a first target sequence and a second target sequence within a test region of a nucleic acid sequence comprising:
conducting a nucleic acid amplification reaction, to form a forward amplicon strand and a reverse amplicon strand of the test region,
contacting the forward amplicon strand with a first probe labelled with a first FRET label and capable of hybridising to the first target sequence of complement thereof in the forward amplicon strand, and
contacting the reverse amplicon strand with a second probe labelled with a second FRET label and capable of hybridising to the second target sequence or complement thereof in the reverse amplicon strand;
wherein the nucleic acid amplification reaction is conducted using a forward amplification primer labelled with a third FRET label and a reverse amplification primer labelled with a fourth FRET label, the forward primer being incorporated into the forward amplicon strand and the second primer being incorporated into the reverse amplicon strand during the amplification reaction;
and further wherein the first and third FRET labels form a first FRET pair and the second and fourth FRET labels form a second FRET pair, each FRET pair comprising a donor label;
the method further comprising the steps of exposing the sample to an excitation source having a wavelength which excites the donor label in the first FRET pair and the donor label in the second FRET pair, detecting fluorescence from the sample and relating this to the presence or absence of the first and second target sequences.

2. Method according to claim 1 wherein the nucleic acid amplification reaction is conducted in the presence of the first and second probes.

3. Method according to claim 1 comprising the step of determining a melting profile of the forward amplicon strand by monitoring fluorescence from the sample at a first wavelength.

4. Method according to claim 3 wherein the presence of a polymorphism in the first target sequence is detected by detection of a different peak melting temperature of the forward amplicon strand compared to the peak melting temperature in a sample not having the polymorphism.

5. Method according to claim 1 further comprising the step of determining a melting profile of the reverse amplicon strand by monitoring fluorescence from the sample at a second wavelength.

6. Method according to claim 5 wherein the presence of a polymorphism in the second target sequence is detected by detection of a different peak melting temperature of the reverse amplicon strand compared to the peak melting temperature in a sample not having the polymorphism.

7. Method according to claim 1 comprising detection of the presence of the first target by detection of a first melting peak when a polymorphism is not present in the first target sequence and by detection of a second melting peak when a polymorphism is present in the first target sequence and comprising detection of the presence of the second target by detection of a third melting peak when a polymorphism is not present in the second target sequence and by detection of a fourth melting peak when a polymorphism is present in the second target sequence.

8. Method according to claim 1 wherein the first FRET label is a fluorescence donor molecule and the third FRET label is a fluorescence acceptor molecule able to absorb fluorescence from the first FRET label, or the third FRET label is a fluorescence donor molecule and the first FRET label is a fluorescence acceptor molecule able to absorb fluorescence from the third FRET label.

9. Method according to claim 1 wherein the second FRET label is a fluorescence donor molecule and the fourth FRET label is a fluorescence acceptor molecule able to absorb fluorescence from the second FRET label, or the fourth FRET label is a fluorescence donor molecule and the second FRET label is a fluorescence acceptor molecule able to absorb fluorescence from the fourth FRET label.

10. Method according to claim 1 wherein the first and second FRET labels are different from one another, and the third and fourth FRET labels are the same as one another.

11. Method according to claim 1 wherein the first and second FRET labels are the same as one another and the third and fourth FRET labels are different from one another.

12. Method according to claim 1 wherein the first and second FRET labels are the same as one another and the third and fourth FRET labels are the same as one another.

13. Method according to claim 1 wherein the nucleic acid sequence comprising the test region is RNA and the method comprises a step of carrying out a reverse transcription reaction.

14. Kit comprising a first nucleic acid probe labelled with a first FRET label, a second nucleic acid probe labelled with a second FRET label, a forward nucleic acid amplification primer labelled with a third FRET label and a reverse nucleic acid amplification primer labelled with a fourth FRET label, wherein the first and third FRET labels form a first FRET pair including a first donor label and the second and fourth FRET labels form a second FRET pair comprising a second donor label, the first and second donor labels being excitable at the same wavelength.

15. Kit according to claim 14 further comprising a DNA polymerase.

16. Kit according to claim 14 wherein the first FRET label is a fluorescence donor molecule and the third FRET label is a fluorescence acceptor molecule able to absorb fluorescence from the first FRET label, or the third FRET label is a fluorescence donor molecule and the first FRET label is a fluorescence acceptor molecule able to absorb fluorescence from the third FRET label.

17. Kit according to claim 14 wherein the second FRET label is a fluorescence donor molecule and the fourth FRET label is a fluorescence acceptor molecule able to absorb fluorescence from the second FRET label, or the fourth FRET label is a fluorescence donor molecule and the second FRET label is a fluorescence acceptor molecule able to absorb fluorescence from the fourth FRET label.

18. Kit according to claim 14 wherein the first and second FRET labels are different from one another, and the third and fourth FRET labels are the same as one another.

19. Kit according to claim 14 wherein the first and second FRET labels are the same as one another and the third and fourth FRET labels are different from one another.

20. Kit according to claim 14 wherein the first and second FRET labels are the same as one another and the third and fourth FRET labels are the same as one another.

21. Kit according to claim 14 further comprising a reverse transcriptase.

22-23. (canceled)
